(19) United States
(12) Patent Application Publication
(10) Pub. No.: US 2022/0213418 A1
(43) Pub. Date: Jul. 7, 2022

(54) METHOD FOR GROWING AQUATIC ORGANISMS CAPABLE OF PHOTOSYNTHESIS IN A CONTROLLED AQUEOUS ENVIRONMENT

(71) Applicant: O&N B.V., Bolsward (NL)

(72) Inventors: Filips Gustaaf Hendrik JAGER, Oosterhesselen (NL); Hendrik Jan HULSHOFF, Oosterhesselen (NL); Dick VAN VELZEN, Oosterhesselen (NL); Jarno STEL, Oosterhesselen (NL)

(73) Assignee: O&N B.V., Bolsward (NL)

(21) Appl. No.: 17/595,126

(22) PCT Filed: May 8, 2020
(86) PCT No.: PCT/NL2020/050296

§ 371 Date: Nov. 9, 2021

(30) Foreign Application Priority Data
May 9, 2019 (EP) 19173537.2

(51) Int. Cl. C12M 1/00; C12N 1/12

(52) U.S. Cl. CPC C12M 21/02; C12N 1/12; C12M 41/06; C12M 31/02

(57) ABSTRACT

The present invention relates to a method for growing aquatic organisms capable of photosynthesis in a controlled aqueous environment and a system for carrying out said method. The invention provides a method and system for growing aquatic organisms capable of photosynthesis under controlled conditions and which uses low energy input.

The present invention relates to a method for growing organisms capable of photosynthesis in a controlled environment and a system for carrying out said method.

Organisms capable of photosynthesis convert light energy into chemical energy by converting carbon dioxide and water into the end-products oxygen and carbohydrates. This process is energized by light.

Algae form an example of such organisms, and are well-known as efficient producers of biomass. During photosynthesis, algae utilize carbon dioxide and light in the presence of water to produce oxygen and biomass. The algae produce lipids, vegetable oils and proteins which can be used for various purposes, for example as a source for human or animal nutrition, pharma or biofuel. Because algae do not have roots, stems and flowers, algal conversion to edible biomass is extremely efficient. Algae grow best under controlled conditions. For instance, algae are sensitive to temperature and light conditions, and in particular fluctuation thereof. Algal yield may be improved by controlling the growth parameters, such as temperature, CO₂ levels, light and nutrients.

The same applies to land cultures such as ground crops, flowering plants and ornamental plants.

The use of artificial light for plant growth is known, for instance by using light emitting diodes (LEDs).

The inventors have found that the present growing systems which make use of artificial light have their limitations with regard to efficiency, especially with regard to the required high energy input.

US 2016/0014974 A1 discloses a stacked culture system for land plants and mentions exposing the plants to light cycles of 3 milliseconds of light followed by 3 milliseconds darkness. US 2016/0014974 A1 does not disclose or suggest any applicability of its disclosure for aquatic culturing, such as algae culture.

EP 0 214 271 A1 discloses a plant for cultivating algae in liquid culture and suggests a relatively weak artificial illumination alternating with relatively long dark periods as favourable. EP 0 214 271 A1 discloses fluorescent lamps as light sources. The light flashes are disclosed to have a duration down to milliseconds. It is further disclosed in EP 0 214 271 A1 that the ratio of the time during which the algae in the liquid are kept in the dark to the time during which they are illuminated is 10:1.

CN 108192801A discloses a dynamic photobioreactor with a culture tank of light transmitting material, allowing natural light to pass through the culture tank and a method of cultivating microalgae using dynamic light, wherein the light quality, light intensity and light and dark frequency can be adjusted. Because in a CN 108192801A a tank of light transmitting material is used which allows natural light to pass it is not possible to fully synchronize the dark periods to which the microalgae are exposed in the tank. Furthermore, CN 108192801A fails to disclose how light quality, light intensity and light and dark frequency can be adjusted.

### SUMMARY OF THE INVENTION

The aim of the invention is to provide a method and system for growing organisms capable of photosynthesis under controlled conditions and which uses low energy input.

In one aspect the invention relates to a method for growing aquatic organisms capable of photosynthesis within a controlled aqueous environment, wherein within said controlled environment a plurality of light sources is arranged, and wherein the inside of said controlled environment is configured such that light from a source outside said controlled environment does not enter said controlled environment; wherein the method comprises controlling said light sources to emit light pulses; exposing the organisms in said controlled environment to said light pulses; and exposing said organisms to synchronised dark periods between light pulses; wherein said light sources are light emitting diodes (LEDs); wherein the synchronised dark periods have a length in the order of milliseconds; and wherein the length of the light pulses is in the order of nanoseconds (1-999 ns) or less.

In a second aspect the invention relates to a system for growing aquatic organisms capable of photosynthesis, comprising a controlled environment, wherein within said controlled environment a plurality of light sources is arranged, and wherein the inside of said controlled environment is configured such that entry of light from a source outside said controlled environment can be completely prevented; and a light source driver, configured to control said light sources to emit light pulses, such that dark periods between light pulses in said controlled environment are in sync; wherein said light sources are light emitting diodes (LEDs); wherein the dark periods have a length in the order of milliseconds; and wherein the length of the light pulses is in the order of nanoseconds (1-999 ns) or less.

The method according to the first aspect can be carried out with the system according to the second aspect.

### DETAILED DESCRIPTION OF THE INVENTION

The present invention is based on the finding that growing organisms capable of photosynthesis under conditions wherein the only source of light comes from light pulses which are controlled such that periods of light are interrupted by periods of complete darkness which highly reduces the amount of energy needed for growth of said organisms.

Photosynthesis is based on the capture of light photons by organisms capable of photosynthesis in order to convert light energy into chemical energy that can be used to generate biomass.

In nature sunlight is the only light source for photosynthesis and is continuously present during day time. However, only a minor fraction of sunlight arriving at and “captured” by illuminated organisms is converted to chemical energy and biomass. In fact, the inventors have observed that photosynthesizing organisms exposed to sunlight only utilize a very limited amount of this light for their activities. Estimations are that less than 13% of the sunlight to which an organism is exposed can be converted to chemical energy and biomass. One reason for this lies in the fact that only light with specific wavelengths can be utilized by the photosynthesis machinery. Another reason is that the kinetics of the photosynthesis mechanism require a certain lag time or down time between consecutive photons to be captured by an individual photosynthesis system. In order to explain this the kinetics of the photosynthesis mechanism will be discussed in summary in the following paragraphs.

Photons emitted from a light source are absorbed by antenna-like structures near the organism's surface which are light-harvesting pigment complexes. The first step of this absorption only takes femto- to picoseconds. Next, excited energy states (so-called excitons) are transferred through so-called inter-protein hopping and magnetic resonance to the reaction centres of the Photosystem II (PSII or reaction centre II or P680), causing the excitation of an electron. This takes 300-500 ps. It also requires two such electrons to be generated in order to change the PSII into reduced, i.e. ‘closed’ state, commonly referred to as P680*. Once PSII is in the ‘closed’ state, further excess photon energy cannot be transferred to or absorbed by the PSII. This then redundant photon energy is released through various energy dissipation mechanisms and is as such lost for photosynthesis. This excess photon energy therefore will not be converted to chemical energy and biomass.

Continuing with the kinetics of the photosynthesis mechanism, the low redox state of P680* reduces primary electron acceptor pheophytin within 3-8 ps, so that PSII becomes oxidized (a state commonly referred to as P680+). Pheophytin then passes on the electron to plastoquinol, starting a flow of electrons down a linear electron transport chain which includes oxidation of plastoquinol by cytochrome b6f and leading eventually to the reduction of NADP to NADPH. This in its turn creates a proton gradient across the chloroplast membrane, which can be used by ATP synthase in the synthesis of ATP. ATP is used as energy source to drive many processes in living organisms. The PSII regains the electron it lost in the beginning of the process when a water molecule was split during a process called photolysis. When PSII regains the electron it returns to the “opened state” and a subsequent photon can be captured again to generate a new exciton.

The slowest (3-5 ms) and therefore limiting step in the linear electron transfer chain is the oxidation of plastoquinol by cytochrome b6f, while the other process steps in the chain require times in the order of picoseconds.

In other words, while it takes only light flashes with a duration in the femto/picosecond scale to produce excitons and thus trigger the photosynthesis cascade (referred herein as “excitation time”), it takes in the order of milliseconds (approximately 3-5 ms) before a given PSII reopens again so that the subsequent exciton capture may take place again (referred herein as “lag time”). During this millisecond lag time excess photon energy contained in the continuing photon stream which cannot be used to produce excitons, is released through energy dissipation mechanisms, such as photo reflection, non-photochemical quenching and heat, and as such is lost for photosynthesis. This excess photon energy therefore will not be converted to chemical energy and biomass. In fact, at the same time, excess exposure of PSII to light may instead lead to so-called photoinhibition. Photoinhibition comprises a series of reactions that inhibit various activities of PSII, leading to a measurable decrease in photosynthesis efficiency. Photoinhibition is caused by factual and potentially long-lasting, damage to PSII due to light exposure. Photoinhibition occurs at all wavelengths. The more PSII is exposed to light, the more damage will occur and, consequently, the more photosynthesis efficiency will be adversely affected.

The provision of the method and system of the invention make it possible to use much less photon energy whilst still making optimal use of the photosynthesis machinery, because it allows to excite the photosynthesis machinery with a short light pulse while avoiding unnecessary and unusable photon energy by the provision of synchronised dark periods between pulses. Given the difference in duration between excitation time (fs/ps range) and lag time (ms) this results in a potential reduction of required light energy in multiple orders of magnitude.

It is essential that the course of growth of the organisms comprises synchronized dark periods between periods of light pulses in order to ensure periods of complete darkness in which all pigments will return to their resting state. In other words, at the end of a synchronized dark period the pigments of the photosynthesis machinery of organisms will all be reset to the opened state so that a short pulse of light will be able to activate a maximal amount, if not all, of the available PSII molecules, thereby triggering a new chain of electron transport which leads to the reduction of NADP to NADPH, which on its turn enables the synthesis of ATP. If there were no synchronized dark periods, it would not be possible to achieve such a synchronized reset of the PSII molecules, leading to loss of photon energy via processes such as photoreflection and/or heat. Moreover, the above mentioned photoinhibition would take place at any moment of no complete darkness. In order to provide such complete darkness, the aqueous environment in which the micro-organisms are cultured is configured such that entry of light from a source outside said controlled environment can be completely prevented. In other words, the sole light source for illumination of the organisms are the LEDs.

As mentioned above the lag time of the photosynthesis machinery and in particular PSII is approximately 3 to 5 ms. It is therefore preferred that the synchronized dark periods in the order of milliseconds (1-999 ms) have a duration of at least 3 ms, more preferably at least 5 ms, in order to allow full reset of all PSII molecules to avoid use of excess photon energy and to prevent the detrimental effects thereof. In a preferred embodiment therefore, the synchronized dark periods have a length in the order of milliseconds. More preferred, the dark periods have a length of at least 3 milliseconds. Most preferred the dark periods have a length of at least 5 milliseconds. On the other hand, in order not to pause photosynthesis the dark periods are preferably not too long. Therefore the dark periods are preferably between 3 and 10 milliseconds, even more preferably between 5 and 10 ms, most preferably about 5 ms.

On the other hand, the excitation time of the photosynthesis machinery, or in other words the time it takes to capture a photon, is very short, namely in the order of femto/picoseconds. Therefore the length of the light pulses should be chosen as short as technically possible. Light sources that are well equipped for emitting very short pulses of light are diode based light sources or light emitting diodes (LEDs).

LEDs are principally capable of effecting very short light pulses. LEDs are easy in use, can be designed to emit light of a specific wavelength and are relatively cheap.

According to the invention the length of the light pulses is in the order of nanoseconds (1 ns-999 ns) or less, for instance in the order of nanoseconds to picoseconds (1-999 ps). For instance the light pulses may have a length of between 10 and 100 picoseconds.

The inventors have found that nanosecond or even sub-nanosecond pulses can be generated by exploiting the transistor avalanche effect for driving LEDs. This allows to generate very short and controlled light pulses.

The avalanche effect works as follows. To produce a very short (pico to nano seconds) pulse of photons it is necessary to create a very fast release mechanism of electricity. When P-type and N-type material in semiconductors come in contact, a depletion region is formed around the P-N junction. The width of the depletion region decreases with the increase in voltage of forward bias, while the depletion region increases in reverse bias condition. At the initial stage the saturation current is independent of the applied voltage. When the voltage is increased in a specific way, a particular point is reached where the junction breaks down leading to an instantaneous heavy flow of current through the P-N junction. This phenomenon occurs because as the voltage increases, the kinetic energy of minority charge carrier also increases. These very fast-moving electrons are colliding with the other atoms to knock off some more electrons from them. These newly excited electrons further release much more electrons from the atoms by breaking the covalent bond and this leads to a considerable increase in the flow of current through the junction in a very small period of time (avalanche). It is possible to make very fast, high energetic electric current to stimulate an intense but short release of photons in LEDs resulting in a light pulse by using an avalanche pulser LED driver.

It is therefore preferred that the light pulses are driven using an avalanche pulser LED driver. Such a driver makes use of an avalanche pulse generators to generate very short and controlled light pulses. Avalanche pulse generators are known in the art of optics or medics, such as for time domain measurements and cancer therapy. These generators are capable of generating electrical pulses in the order of nanoseconds (1 ns-999 ns) or less, for instance in the order of nanoseconds to picoseconds (1-999 ps). See for instance Veledar et al., 2005, Review and Development of Nanosecond Pulse Generation for Light Emitting Diodes, Corpus ID: 45189079; or Dutta, S. et al, 2017, The avalanche-mode superjunction LED. IEEE Transactions on Electron Devices, 64(4), 1612-1618. Avalanche pulse generators are commercially available and can be configured to the wishes of the user to be used for driving LED pulses with suitable soft and hardware in order to control said LEDs to emit light pulses such that dark periods between light pulses in the bioreactor are in sync.
The inventors have now surprisingly found that such generators can be configured to create pulsar LED drivers to be used in the cultivation of aquatic photosynthetic organisms.

By way of an example, by using such an avalanche pulser LED driver with an avalanche pulse generator the inventors are able to apply a light regimen with cycles of 148 ns light followed by 4 ms of dark. The ratio of the time during which the algae in the liquid are kept in the dark to the time during which they are illuminated in this example is 27000:1, while still having optimal photosynthesis.

This way the invention applies a light regimen to the aquatic photosynthesizing organisms that takes into account the time limitation for causing the P-N jump (when free electrons from N-side and holes from P-side recombine with the opposite charge carriers) of the LEDs and the time limitation of the photosystem of the organism, so that a light regimen is achieved with very low energy input requirements while obtaining optimal photosynthesis and thus optimal growth of the photosynthesizing organisms.

Photosynthesizing organisms are only capable of using specific wavelength fractions from the natural light spectrum for photosynthesis. The spectrum used by organisms capable of photosynthesis is conventionally referred to as the Photosynthetically Active Radiation (PAR). The PAR region comprises wavelengths between 400-700 nm, which corresponds closely to the visible spectrum. Photosynthesis only occurs within the 400-500 and 600-700 nm range in the PAR region, so a large amount of light energy falls outside the PAR region and thus remains unusable. The exact optimal wavelengths differ per species of organism. For instance for most microalgae the major wavelengths usable for photosynthesis are within the range of 420-470 nm (blue) and/or 660-680 nm (red). The exact preference of wavelength and pulse length varies per species. In particular LEDs can be easily designed to emit a specific wavelength in accordance with methods known in the art. It is therefore preferred that said plurality of light sources comprises different types of light sources, each configured to emit light at a predetermined wavelength and pulse length in a synchronised manner, in particular with the values described above. In this case the light source driver is configured to control separately each type of light source to emit light pulses of a predetermined wavelength and pulse length. It is therefore possible to use multiple light sources for emitting light of multiple pulse lengths, as long as there are periods of complete darkness between consecutive pulses. Such a period of darkness may for instance be between the end of a pulse of a particular wavelength and the beginning of a pulse of another wavelength. A pulse of a particular length for a certain wavelength may for instance fall (time wise) within a pulse of a longer length for another wavelength. In the latter case the period of complete darkness will then be between consecutive pulses of the longer length. It can also be that first LEDs emitting a specific wavelength are configured to emit pulses in particular intervals, while further LEDs emitting another specific wavelength skip one or more of said intervals and are configured to emit a pulse coinciding with pulses of the first LEDs, but in a lower frequency.

The invention is applied to aquatic organisms. In this case the organisms are grown in an aqueous medium. In this case said organisms are preferably algae, such as microalgae. In case of aquatic organisms the controlled environment is preferably an bioreactor. When in operation the bioreactor is filled with an aqueous medium containing organisms, for instance algae. In that case the system of the invention is a bioreactor for growing organisms capable of photosynthesis in an aqueous medium, wherein within said bioreactor a plurality of light sources is arranged and wherein the inside of said bioreactor is configured such that entry of light from a source outside said bioreactor can be completely prevented; and a light source driver, configured to control said light sources to emit light pulses, such that dark periods between light pulses in the bioreactor are in sync. In a preferred embodiment such a bioreactor comprises multiple light bars comprising a plurality of distributed LEDs, the bars extending in the inside of the bioreactor. This way as much algae as possible will be exposed to the emitted light, which is advantageous for photosynthesis and thus the production of biomass.

1. A method for growing aquatic organisms capable of photosynthesis within a controlled aqueous environment comprising one or more aquatic organisms, said controlled environment comprising a plurality of light sources arranged within,
the method comprising the steps of:
providing a controlled aqueous environment, the controlled aqueous environment comprising one or more aquatic organisms;
providing a plurality of light sources, the plurality of light sources being arranged within the controlled aqueous environment;
controlling the plurality of light sources to emit light pulses;
exposing the one or more aquatic organisms in the controlled aqueous environment to the light pulses; and
exposing the one or more aquatic organisms to synchronised dark periods between light pulses,
wherein the inside of the controlled aqueous environment is configured such that light from a source outside the controlled aqueous environment cannot enter the controlled aqueous environment,
wherein the plurality of light sources are light emitting diodes (LEDs),
wherein the synchronised dark periods have a length of between 1 and 999 milliseconds, and
wherein the light pulses have a length of less than 1 microsecond.

2. The method according to claim 1, wherein the dark periods have a length of at least 3 milliseconds.

3. The method according to claim 2, wherein the dark periods have a length of at least 5 milliseconds.

4. The method according to claim 1, wherein the plurality of light sources comprises different types of diode based light sources, each different type configured to emit light at a predetermined wavelength and pulse length.

5. The method according to claim 1, wherein the light pulses are controlled by means of an avalanche pulser LED driver.

6. The method according to claim 1, wherein the controlled aqueous environment is a bioreactor, and wherein the one or more aquatic organisms are algae.

7. A system for growing aquatic organisms capable of photosynthesis, comprising
a controlled environment, wherein the controlled environment comprises a plurality of light sources arranged within, and wherein the inside of the controlled environment is configured such that entry of light from a source outside the controlled environment can be completely prevented,
and a light source driver, configured to control the plurality of light sources to emit light pulses, such that dark periods between light pulses in the controlled environment are in sync, and further configured to control the plurality of light sources to emit light pulses such that the dark periods have a length of between 1 and 999 milliseconds and such that the light pulses have a length of less than 1 microsecond;
wherein the plurality of light sources are light emitting diodes (LEDs).

8. The system according to claim 7, wherein the light source driver is an avalanche pulser LED driver.

9. The system according to claim 7, wherein the plurality of light sources comprises different types of light sources, each different type configured to emit light at a predetermined wavelength and pulse length and wherein the light source driver is configured to control separately each different type of light source to emit light pulses of a predetermined wavelength and pulse length.

10. The system according to claim 7, wherein the controlled environment is a bioreactor, and wherein the controlled environment further comprises one or more organisms.

11. The system according to claim 10, wherein the the plurality of light sources comprises a plurality of light bars each comprising a plurality of distributed LEDs, the plurality of light bars extending in the inside of the bioreactor.

12. The method according to claim 6, wherein the one or more aquatic organisms are microalgae.

13. The method according to claim 10, wherein the one or more organisms are algae.

14. The method according to claim 13, wherein the one or more organisms are microalgae.
